(19) United States
(12) Patent Application Publication
(10) Pub. No.: US 2007/0212412 A1
(43) Pub. Date: Sep. 13, 2007

(54) STABLE SOLID FORMULATION OF SERTINDOLE

(75) Inventors: Flemming Enok Olsen, Allerod (DK); Anne Ravnholdt Christensen, Hedehusene (DK); Ken Liljegren, Vaerlose (DK)

Correspondence Address: DARBY & DARBY P.C., P.O. BOX 770, Church Street Station, New York, NY, 10008-0770, US

(73) Assignee: H. Lundbeck A/S, Valby-Copenhagen (DK)

(21) Appl. No.: 11/530,404

(22) Filed: Sep. 8, 2006

Related U.S. Application Data
(63) Continuation-in-part of application No. 11/530,243, filed on Sep. 8, 2006, now abandoned.
(60) Provisional application No. 60/715,787, filed on Sep. 8, 2005.

(51) Int. Cl. A61K 9/20; A61K 31/405

(52) U.S. Cl. 424/464; 514/419

(57) ABSTRACT

The present invention relates to a pharmaceutical composition comprising sertindole and a protective agent absorbing radiation with a wavelenght below 400 nm. The present invention also relates to the use of sertindole for the preparation of a pharmaceutical composition comprising sertindole and a coating layer compsrising said protective agent, such as ironoxide, for the treatment of schizophrenia.

This application claim the benefit of U.S. Provisional Application No. 60/715,787 filed Sep. 8, 2005, which is hereby incorporated by reference.

The present invention relates to a pharmaceutical composition comprising sertindole and a protective agent absorbing radiation with a wavelength below 400 nm, such as iron oxide. The present invention also relates to a solid pharmaceutical composition comprising sertindole and a coating layer comprising a protective agent absorbing radiation with a wavelength below 400 nm. Furthermore, it relates to use of sertindole for the preparation of a pharmaceutical composition, as well as use of iron oxide for the preparation of a coating composition.

### BACKGROUND

Sertindole, chemically named 5-chloro-1-(4-fluorophenyl)-3-(1-(2-(2-imidazolinon-1-yl) ethyl-4-piperidyl-1H-indole, is an antipsychotic drug with high affinity for serotonin 5-HT₂, dopamine D₂ and α₁-adrenergic receptors. Sanchez et al., Drug Dev Res. 1991; 22:239-250; Arnt J and Skarsfeldt T, Neuropsychopharmacol. 1998; 18(2):63-101. Sertindole is disclosed in U.S. Pat. No. Re. 34,299, and its antipsychotic activity is disclosed in U.S. Pat. No. 5,112,838. A method of manufacturing sertindole is disclosed in U.S. Pat. No. 6,335,463. Another method of manufacturing sertindole is disclosed in WO 03/080597.

Most research directed at the therapeutic effectiveness of sertindole has focused on its use in the treatment of schizophrenia. See, e.g., U.S. Pat. No. 5,112,838; Brown et al., Pharmacotherapy. 1993; 18(1):69-83; Samara, E. and Granneman, R., Clin. Pharmacol. & Therapeutics. 1996; 59(2):187; and Tamminga et al., International Clin. Psychopharmacol. 1997; 12(suppl. 1):S29-S35. Sertindole may also be effective in the treatment of other disorders such as: psychosis, including drug induced psychosis (U.S. Pat. No. 5,238,945); anxiety (U.S. Pat. No. 5,439,922); memory impairment (U.S. Pat. No. 5,444,073); substance dependency (U.S. Pat. No. 5,462,948); and depression, hypertension, and extrapyramidal side effects of other antipsychotic drugs (U.S. Pat. No. 5,703,087).

During the clinical development programme, sertindole was demonstrated to have effect against both positive and negative symptoms of schizophrenia. It was well-tolerated and showed placebo-level incidence of extrapyramidal side effects. These properties may lead to improved patient compliance with treatment and a consequent decrease in relapse rate—a problem widely recognised as one of the greatest challenges facing psychiatric medicine.

### DESCRIPTION OF THE INVENTION

The present inventors have discovered that sertindole (manufactured by H. Lundbeck A/S, Copenhagen-Valby, Denmark) (Serdolect®, Zerdol®) undergoes a light-induced degradation process when formulated as film-coated tablets, such as tablets comprising 2 mg, 4 mg, 8 mg, 12 mg, 16 mg, 20 mg and 24 mg of sertindole (calculated as the free base) coated with a white film-coating layer, containing titanium dioxide as the white pigment, in amounts corresponding to 2-4% film-layer relative to the tablet core weight, indicating that such tablets are sensitive to light.

The effect of light protection with increasing amount of titanium dioxide in the film-coating layer on a tablet formulation containing sertindole has been analysed. White film suspension offers little or almost no protection against light with wavelength below 400 nm up to 3% of film-layer.

The effect of light protection with increasing amount of iron oxides in the film-coating layer on a tablet formulation comprising sertindole has been analysed.

During the pharmaceutical development of coloured film-coated sertindole tablets it was revealed that the tablet cores were protected against the detrimental effect of light when including iron oxide, such as red, yellow, or black, in mixture or alone, in the coating.

Moreover, the light stability studies have revealed that the levels of at least three impurities (see below figure) are increased during the light-induced degradation process.

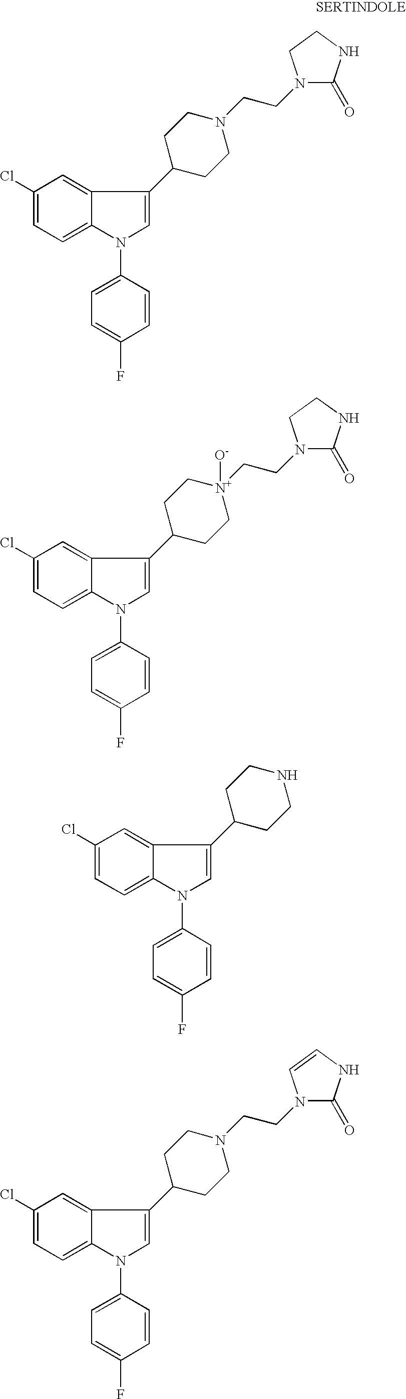

According to the inventors, and without limiting the present invention, including iron oxide, carbon black, or indigocarmine pure or as aluminium lake, in a coating, such as the coating of a tablet pellets or capsule; mixed in the composition core, such as a tablet or capsule; or in the container wall; either as an outside layer or mixed in the container wall material (e.g. glass or organic polymeric material container) or in the primary or secondary packaging material comprising sertindole as a liquid, such as a vial or dropping bottle comprising iron oxide, or indigocarmine, would protect sertindole against the detrimental effect of light.

Typically, a light-protective coating is highly interesting since it obviates the need for special precautions against exposure to artificial or natural light after the coating process, packaging and storage of bulk product, such as tablets, as well as finished product (e.g. drug product in final packaging).

Typically, a light-protective composition core comprising a mixtures of iron oxide, black carbon or indigocarmine as aluminium lake and sertindole is highly interesting since it obviates the need for special precautions against exposure to artificial or natural light during the processing, packaging and storage of bulk product, such as tablets, as well as finished product (e.g. drug product in final packaging).

Typically, a light-protective container is highly interesting since it obviates the need for special precautions against exposure to artificial or natural light after filling and storage of bulk product, such as drops and other liquid oral formulations, as well as finished product (e.g. drug product in final packaging).

Furthermore special considerations as to choice of packaging material (primary as well as secondary) are no longer required. Standard packaging material can now be used.

In addition, in respect of solid compositions, it is now possible to differentiate between dosage strengths by colours. Different dosage strengths are needed due to the need of titration of dosage from lowest strength (typically 4 mg) to the strength where optimum effect is obtained (typically 12-20 mg). Different coloured tablets or capsules mitigate risk of intake of wrong dosage, and ease communication on dosage between prescriber, treatment personnel, and patient.

Sertindole has the general formula: 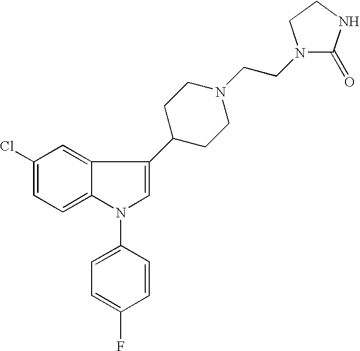
and the chemical name 5-chloro-1-(4-fluorophenyl)-3-(1-(2-(2-imidazolinon-1-yl) ethyl-4-piperidyl-1H-indole, and throughout the description “sertindole” is intended to include any form of the compound, such as the base, pharmaceutically acceptable salts, e.g. pharmaceutically acceptable acid addition salts, hydrates or solvates of the base or salt, as well as anhydrates, and also amorphous, or crystalline forms.

The pharmaceutically acceptable acid addition salts of the compound may be formed with non-toxic organic or inorganic acids in an aqueous miscible solvent, such as acetone or ethanol, with isolation of the salt by concentration and cooling or an excess of the acid in aqueous immiscible solvent, such as ethyl ether or chloroform, with the desired salt separating directly.

Exemplary of such organic salts are those with maleic, fumaric, benzoic, ascorbic, embonic, succinic, oxalic, bis methylene-salicylic, methanesulfonic, ethanedisulfonic, acetic, propionic, tartaric, salicylic, citric, glucomic, lactic, malic, mandelic, cinnamic, citraconic, aspartic, stearic, palmitic, itaconic, glycolic, p-amino-benzoic, glutamic, benzene sulfonic and theophylline acetic acids as well as the 8-halotheophyllines, for example 8-bromo-theophylline. Exemplary of such inorganic salts are those with hydrochloric, hydrobromic, sulfuric, sulfamic, phosphoric and nitric acids. Of course, these salts may also be prepared by the classical method of double decomposition of appropriate salts, which is well-known to the art.

When it is desired to isolate sertindole in the form of the free base, this may be done according to conventional procedure, such as by dissolving the isolated or un-isolated salt in water, treating with a suitable alkaline material, extracting the liberated free base with a suitable organic solvent, optionally drying the extract with a suitable drying agent prior to evaporating the extract to dryness to effect isolation of the free basic amine. The extract may optionally be subjected to fractional distillation.

Iron oxides are commercially available from different suppliers, e.g. BASF or Univar Export, and comprise Fe₂O₃ or FeO as well as mixtures thereof. Throughout the description “iron oxide” is intended to comprise any form thereof, such as without limitation yellow iron oxide (Fe₂O₃ , H₂O), red iron oxide (Fe₂O₃), black iron oxide (FeO,Fe₂O₃), as well as mixtures thereof.

As used herein, a pharmaceutical composition is intended for oral administration and include solid dosage forms (also referred to as a solid pharmaceutical composition) such as capsules, tablets, orodispersible tablets, dragees, pills, lozenges, powders and granules, semisolids and liquid dosage forms (also referred to as a liquid pharmaceutical composition) such as solutions, emulsions, suspensions, syrups and elixirs. Where appropriate, they can be prepared with coatings such as enteric coatings, film-coatings, sugar coatings, powder coatings and compression coating. The solid dosage form of sertindole may optionally be coated with functional and/or non-functional layers comprising film-forming polymers, if desired. Suitable film-forming polymers include one or more of ethyl cellulose, hydroxypropylmethylcellulose, hydroxypropylcellulose, methylcellulose, carboxymethylcellulose, hydroxymethylcellulose, hydroxyethylcellulose, cellulose acetate, hydroxypropylmethylcellulose phthalate, cellulose acetate phthalate, cellulose acetate trimellitate; waxes such as polyethylene glycol; methacrylic acid polymers such as Eudragit®RL and RS; and the like. Alternatively, commercially available coating compositions may be used for the coating, including film-forming polymers marketed under various trade names such as, Opadry®. The solid dosage form of sertindole may optionally be coated with functional and/or non-functional layers comprising plasticizers within film-forming polymers, if desired. Suitable plasticizers include one or more of polyethylene glycol, triethyl citrate, triacetin, acetyl triacetylcitrate, acetyltributyl citrate, dibutyl phthalate, dibutyl sebacate, diethyl phthalate, tributyl citrate.

Film-Layer=Film-Coating Layer (Dry).

Film-layer in % corresponds to percent weight of film-layer relative to weight of tablet core. (Film-layer mg. on one tablet/weight of one tablet mg.)*100

Determination of Amount of Film-Layer

Applied film-layer may be determined by one of following methods:

Weighing—determination of weight gain on a representative sample (sample size:10-1000 tablets) after the coating process.

Determination of film-coating layer by NIR

Chemical determination of amount of (pigment/colorant) followed by calculation of amount of film-layer, by the ratio of pigment in the film-coating layer.

Pigment

Amount of pigment (e.g. iron-oxide, indigocarmine, indigocarmin-A1-lake, other colorants^{A} absorbing light of wavelength<400 nm, metals or mixture of metals absorbing light of wavelength<400 nm, salts of metals or mixture of metal salts absorbing light of wavelength<400 nm )

^{A} Permanently or provisionally listed colour additives subject to U.S. certification in 2000 (Based on 21 CFR 2000).

Pigment may be Expressed:

Either as

Percentage of Suspension.

Pigment as % of suspension. (gram pigment/kg suspension)/100.

Percentage of Dry Film-Layer.

Pigment as % of film-layer. (mg pigment on one tablet/mg film-layer on one tablet)*100.

Percentage of Tablet-Core or Capsule Content.

Pigment as % of core-weight or weight of capsule content (mg pigment in one tablet-core of capsule content/weight (mg) of one tablet-core or weight (mg) of content of one capsule)*100.

Microgram (μg) per Square Millimetre (mm²) Tablet Surface Area.

Pigment μg/mm² tablet.

Microgram (μg) per Square Millimetre (mm²) Capsule Surface Area.

Pigment μg/mm² capsule.

Microgram (μg) per Square Millimetre (mm²) Primary Container Surface Area.

Pigment μg/mm² container

Microgram (μg) per Square Millimetre (mm²) Packaging Material Surface Area (PMSA).

Pigment μg/mm² PMSA.

Where appropriate, the pharmaceutical composition of the invention may also comprise pharmaceutical carriers such as inert solid diluents or fillers, sterile aqueous solution and various organic solvents. Examples of liquid carriers are syrup, peanut oil, olive oil, phospholipids, fatty acids, fatty acid amines, polyoxyethylene and water. Examples of solid carriers are lactose, terra alba, sucrose, cyclodextrin, talc, agar, pectin, acacia, stearic acid and lower alkyl ethers of cellulose corn starch, potato starch, talcum, magnesium stearate, gelatine, lactose, gums, and the like. Any other adjuvants or additives usually used for such purposes such as colourings, flavourings, preservatives etc. may be used provided that they are compatible with the active ingredients.

Accordingly, and in one aspect, the present invention relates to a pharmaceutical composition comprising sertindole and a protective agent absorbing radiation with a wavelength below 400 nm, such as iron oxide, or indigocarmine, or other pigments mentioned herein.

In a further aspect, the present invention relates to a solid pharmaceutical composition comprising sertindole and a coating layer comprising a protective agent absorbing radiation with a wavelength below 400 nm. In an embodiment the solid pharmaceutical composition is selected from tablets and capsules. Typically, the amount of sertindole in the solid pharmaceutical composition, such as tablets and capsules, is from 1 mg to 48 mg mg, such as 2 mg to 24 mg, e.g. 2 mg, 4 mg, 8 mg, 12 mg, 16 mg, 20 mg and 24 mg.

Typically, a coating suspension is made by mixing a dry powder comprising a polymer, such as a film-forming polymer, a plasticizer, optionally a second film-forming polymer, optionally a pigment, such as titanium dioxide, and optionally an anti-caking agent, and then mixing with water and optionally adding an anti-coalescing agent to prevent the coating suspension from clogging the nozzles and lines of the spray gun apparatus.

Examples of coating compositions from “Aqueous Polymeric Coatings for Pharmaceutical Dosage Forms”, Drugs and the pharmaceutical Sciences Vol. 79, Second Ed., Marcel Dekker Inc., Edited by James W. McGinity.

| Component | Formula 1 | Formula 2 | Formula 3 |
|---|---|---|---|
| Eudragit NE 30D^{a} | 15.0 | — | — |
| Eudragit RL 30 D^{a} | — | 5.0 | — |
| Eudragit RS 30 D^{a} | — | — | 11.5 |
| PEG 6000 | — | 0.5 | — |
| Triethyl Citrate | — | 1.0 | 2.2 |
| Talc | 7.5 | 8.5 | 5.8 |
| Pigments incl. TiO₂ | — | 5.0 | — |
| Water | 77.5 | 80.0 | 80.5 |
| | 100.0 | 100.0 | 100.0 |
| Applied coating, | 1-5 | 1-2 | 2-5 |
| mg polymer/cm² | | | |

^{a}30% dry substance

| Component | Amount |
|---|---|
| HPMCAS (AS_MF) | 7.00% |
| Triethyl citrate | 1.96% |
| Sodium lauryl sulfate | 0.21% |
| Talc | 2.1% |
| Sorbitan sesquioleate | 0.0025 |
| Water | 88.73% |

| Component | Amount |
|---|---|
| Hydroxypropylmethylcellulose (pharmacoat 606) | 6% |
| Titanium dioxide | 0.5% |
| Water | 93.5% |

| Kollidon VA 64 | 5 | g |
|---|---|---|
| Ethocel ® 20 | 5 | g |
| Titanium dioxide | 20 | g |
| Talc | 13 | g |
| Colour lake | q.s. | |
| Isopropanol | 98 | g |
| Water | ad 200 | g |

| | | |
|---|---|---|
| Sucrose | 40 | g |
| Kollidon VA 64 | 10 | g |
| Lutrol E 400 | 8 | g |
| Colour lake | 3 | g |
| Titanium dioxide | 6 | g |
| Talc | 10 | g |
| Water | ad 240 | g |

Alternatively, commercially available coating compositions (such as Opadry® may be used and mixed with the protective agent absorbing radiation with a wavelength below 400 nm, such as iron oxide, and then applied to the surface of the solid pharmaceutical composition, such as tablets or capsules. Typically, the coating layer is provided onto the tablets or capsules by spraying.

Typically, the protective agent absorbing radiation with a wavelength below 400 nm is present in an amount sufficient to protect the sertindole from light degradation, and such protective agent is without limitation selected from iron oxide, such as yellow, red, or black iron oxide as well as mixtures thereof. Alternatively, the protective agent absorbing radiation with a wavelength below 400 nm is selected from Carbon black, indigocarmine, indigocarmine-aluminium lake, other colorants (Permanently or provisionally listed color additives subject to U.S. certification in 2000 (Based on 21 CFR 2000) absorbing light of wavelength<400 nm, metals or mixture of metals absorbing light of wavelength<400 nm, salts of metals or mixture of metal salts absorbing light of wavelength<400 nm.

The protective agent, such as iron oxide, is preferably distributed evenly in the coating layer. Typically, the coating layer is less than 20% weight relative to the core of the composition, and for processing, handling and convenience, it is less than 10% weight, such as 0.5% to 10%, typically, the coating layer is 0.5-4% by weight.

The coating layer comprising the protective agent may be a first or second or further coating layer, thus for instance, a tablet core may be coated with a sugar coating, and then with the coating layer comprising the protective agent, or vice versa. Typically, the coating layer comprising the protective agent is a final coating layer.

Typically, when the coating layer comprises iron oxide as the protective agent, such as iron oxide is present in an amount of at least 0.1 μg/mm² surface area of the composition. This is protecting the sertindole from the detrimental effect of light. Alternative amounts of iron oxide in the coating layer is selected from at least 0.15 μg/mm², at least 0.2 μg/mm², at least 0.25 μg/mm², at least 0.3 μg/mm² or at least 0.35 μg/mm² surface area of the composition. The inventors have shown that iron oxide present in the coating layer in an amount of above 0.4 μg/mm² surface area of the composition is just as effective as a dark control. However, this is not intended to limit the invention in any way.

Additionally, the coating layer may comprise titanium dioxide. Titanium dioxide in an amount of about 30 weight % of dry substance in the coating layer corresponds to approximately 12 hours in the coating process to obtain a comparable light protecting effect to iron oxides and is considered inconvenient, on the other hand it is desirable to include titanium dioxide in the coating layer of at least 2 weight % of dry substance in the coating layer.

In an embodiment the coating layer comprises a film-forming polymer, such as one or more of ethyl cellulose, hydroxylpropylmethylcellulose, hydroxypropylcellulose, methylcellulose, carboxymethylcellulose, hydroxymethylcellulose, hydroxyethylcellulose, cellulose acetate, hydroxypropylmethylcellulose phthalate, cellulose acetate phthalate, cellulose acetate trimellitate; waxes such as polyethylene glycol; methacrylic acid polymers such as Eudragit®RL and RS; and the like.

A conventional coating layer may contain sugar and/or calcium carbonate and/or talc and/or titanium dioxide.

Hard or soft capsules consist of gelatine, microcrystalline cellulose or starches.

In the solid pharmaceutical composition as described above in any one of the embodiments, the coating layer may comprise a plasticizer, for instance, to soften the polymer. Exemplary of such plasticizer is one or more of polyethylene glycol (molecular weight form 200 to 8000, e.g. Carbowax by Union Carbide), glycerine, propylen glycol, acetylated monoglyceride, triethylcitrate, Triacetin (available from Pfizer), acetyl triacetylcitrate, acetyl tributyl citrate, dibutyl phthalate, dibutyl sebacate, diethyl phthalate, tributyl citrate. Typically, polyethylenglykol 400 (PEG 400) is used as the plasticizer.

The study as described in the experimental part was originally planned to be performed with film-coating suspensions containing PEG 400 corresponding to 12 weight % in the solid film coating layer. Increasing amount of degradation, after light stress testing, was found, when increasing amount of film coating layer from film suspension containing yellow iron oxide and PEG 400 corresponding to 12 weight % in the solid film-layer was applied. No negative effect of the plasticizer, PEG 400, were seen in amounts from above 0 weight % to about 8 weight % of the film coating layer, however at a level of 12 weight % PEG 400 a reduction in light protection effect was observed.

Thus, in a further embodiment the coating layer comprises a polyethylene glycol in an amount of less than 12 weight % relative to the coating layer, such as less than 8 weight %, less than 6 weight %, less than 4 weight %, typically from above 0 weight % to 8 weight %.

Another alternative of the present invention is preparation of a mixture comprising sertindole and a protective agent absorbing radiation with a wavelength below 400 nm, and wherein said mixture is formulated into a solid pharmaceutical composition.

Accordingly, in a further aspect the present invention relates to a solid pharmaceutical composition comprising a mixture of sertindole and a protective agent absorbing radiation with a wavelength below 400 nm, such as iron oxide, or indigocarmine as aluminium lake. In an embodiment the solid pharmaceutical composition is selected from tablets and capsules. Typically, the amount of sertindole in the solid pharmaceutical composition, such as tablets and capsules, is from 1 mg to 48 mg, such as 2 mg to 24 mg, e.g. 2 mg, 4 mg, 8 mg, 12 mg, 16 mg, 20 mg and 24 mg.

Typically, the protective agent absorbing radiation with a wavelength below 400 nm is present in an amount sufficient to protect the sertindole from light degradation, and such protective agent is without limitation selected from iron oxide, such as yellow, red, or black iron oxide as well as mixtures thereof. Alternatively, the protective agent absorbing radiation with a wavelength below 400 nm is selected from carbon black, or indigocarmine, alone or linked to aluminum.

In a further embodiment the protective agent absorbing radiation with a wavelength below 400 nm is present in an amount of 0.1% to 5% relative to the weight of the solid composition.

In a further embodiment the solid pharmaceutical composition, such as tablet or capsule, further comprises a coating layer, such as a film-coating layer. A typical coating layer is described above in relation to the coating comprising iron oxide.

In a further embodiment the coating layer comprises iron oxide, such as iron oxide in an amount of at least 0.1 μg/mm² surface area of the composition. A particular embodiment is the solid pharmaceutical composition comprising iron oxide mixed together with sertindole as well as a coating layer comprising iron oxide.

In a further aspect, the present invention relates to a liquid peroral pharmaceutical composition consisting of a container means comprising sertindole in a liquid formulation, wherein said container means comprises a protective agent absorbing radiation with a wavelength below 400 nm, such as iron oxide.

In a further aspect the present invention relates to use of sertindole for the preparation of a pharmaceutical composition for the treatment of schizophrenia, wherein the composition comprises sertindole and a protective agent absorbing radiation with a wavelength below 400 nm, such as iron oxide.

In a further aspect the present invention relates to a method of treating a subject in need thereof, comprising administering a pharmaceutical composition for the treatment of schizophrenia, wherein the composition comprises sertindole and a protective agent absorbing radiation with a wavelength below 400 nm, such as iron oxid. In an embodiment sertindole is administered in an amount of 2-24 mg once daily.

In a further aspect the present invention relates to use of iron oxide for the preparation of a coating composition comprising a protective agent absorbing radiation with a wavelength below 400 nm, such as iron oxide.

In a further aspect the present invention relates to a coating composition comprising a protective agent absorbing radiation with a wavelength below 400 nm, such as iron oxide, a polymer, and a plasticizer. In an embodiment the coating composition comprises iron oxide and Opadry®.

In the following, the invention is illustrated by way of examples. However, the examples are merely intended to illustrate the invention and should not be construed as limiting.

Experimental Section

Materials and Equipment

Materials:

The following ingredients were used:

Sertindole (as free base) (H. Lundbeck A/S), Maize Starch (Maize starch B, Roquette, Lestrem Cedex, France), Lactose monohydrate (Pharmatose 350 M,DMV International, Veghel.Netherland), Hydroxypropylcellulose (Klucel EXF Pharm,

Hercules, Virginia, U.S.A), Magnesium stearate, (Liga magnesium Stearate MF-2-V, Akcros Chemical, Venlo, Netherland), Microcristalline Cellulose (Avicel PH-102,FMC International, Cork, Ireland), Croscarmellose Sodium, (Ac-Di-Sol, FMC International, Cork, Ireland), Iron Oxide yellow, red and black, (Univar Export, Essex, England) Polyethylene glycol 400, (Polyglykol 400, Clariant GmbH, Gendorf, Germany), Titanium dioxide, (1700 Titanium dioxide, Univar Export, Essex England) Hypromellose (Methocel E5 premium LV EP, (Dow Chem. Company, Louisiana, U.S.A).

Equipments:

The following apparatus were obtained from the indicated sources:

Diosna P 250 (Diosna and Soehne, Osnabruck, Germany), tray oven Lytzen type BB (Lytzen and Co Herlev, Denmark), sieve Frewitt type MGM 284 (Frewitt Fabrique, Fribourg, Switzerland), Blender Bohle PM100 (L. B. Bohle GmbH, Ennigerloh, Germany), Kilan T 300 (IMA, Kilian Gmbh & Co, Cologne, Germany).

Labcoat IIX ( Ohara technologies, Toronto, Ontario, Canada). Drum 15″.

### EXAMPLE 1

Preparation of Tablets

The formulation composition comprising sertindole 8 mg/tablet in this study is given below: (Batch size: 800.000 tablets).

TABLE 1

| | % in formulation |
|---|---|
| Ingredient | w/w |
| Sertindole | 5.3 |
| Lactose monohydrate | 49 |
| Maize starch | 25 |
| Hydroxy propyl cellulose | 2 |
| Microcrystalline Cellulose | 16 |
| Croscarmellose Sodium | 2 |
| Magnesium stearate | 0.7 |

Composition of sertindole tablets 8 mg
Granulation, Drying and Blending

Sertindole, maize starch, lactose monohydrate, and hydroxyl propyl cellulose are mixed in a Diosna P 250 for 3 minutes at high speed (mixer II/ impeller II).

Purified water (q.s.) is added during 1 min. (mixer and impeller at low speed (I/I)).

Finally granulate is formed during 3 minutes at low speed (II/II).

The wet granulate is passed though a sieve (screen size 4.0 mm) before drying in a tray drying oven.

The granulate is dried on trays by direct heating (static solid bed) at a set point of 50° C. until equilibrium relative humidity is between 25-45%.

The dried granulate is oscillated together with microcrystalline cellulose and croscarmellose sodium through a screen size 1.000 mm.

The powder is blended in the bin blender for 6 minutes at 6 rotations per minute (rpm).

Magnesium stearate prior sieved (0.8 mm) is added and blended for 3 min. at 6 rpm.

Compression

Tablets are compressed on toolings: 6.5 mm×9.5 mm oval punch curved for coating with embossing “S8” at the upper tablet surface.

Compression speed: about 200.000 tablets/hour

Tablet weight: 150 mg

Tablet Height: 3.3 mm

Surface area of one tablet: 127 mm²

Hardness: About 60 N (measured in the longitude direction)

Disintegration time: about 2 minutes

Coating of Tablets

3 coating processes with repetition (i.e. 6 coatings) were performed, each comprising 3 kg of core tablets. 3 different coating suspensions formulae, all containing yellow iron oxide, were included. The plastiziser, Polyethylenglycol 400, were added at following levels: 0 %, 4% and 8% of the dry solid film-layer.

As reference 1 coating was performed on 3 kg of tablet cores with a white film-coating (i.e. without yellow iron oxide). Amount of plastiziser in the white coated tablets formula D was 4%.

Formula of film-coating suspensions see table 2.

Batch size of film-coating suspensions: 3000 g:

TABLE 2

| | | | | | | Batch |
|---|---|---|---|---|---|---|
| | Purified | Hypromellose | Titanium | Polyethylene | Iron | Coated |
| Formula | Water | 5 cps | Dioxide | glycol 400 | oxide Yellow | Tablets |
| A | 90.8%* | 5.87% | 3.14% | 0% | 0.19% | Exp. 1 |
| | | | | | | Exp. 2 |
| B | 90.8%* | 5.87% | 2.77% | 0.37% | 0.19% | Exp. 3 |
| | | | | | | Exp. 4 |
| C | 90.8%* | 5.87% | 2.41% | 0.74% | 0.19% | Exp. 5 |
| | | | | | | Exp. 6 |
| White | 90.8%* | 5.87% | 2.96% | 0.37% | 0% | Exp. 7 |
| Ref. D | | | | | | |

Experimental numbers and composition of film-coating suspensions (content of individuals shown as percentage w/w of actual batchsize of suspension).
*Purified water evaporates during the coating process.

Coating parameters:

Drum: 15″

Temperature inlet: about 60° C.

Temperature outlet: about 46° C.

Drum: about 12 rpm

Air flow: about 560 m³/h

Susp. Flow rate: about 30 g/min

Atomizer Pressure: 3.4 bar

Film coating suspension applied: 1027 g

Sampling (During the Coating Process)

TABLE 3
Amount of dry substance applied to core tablets (% w/w)

| Sample | Sample | Sample | Sample | | | |
|---|---|---|---|---|---|---|
| 1 | 2 | 3 | 4 | Sample 5 | Sample 6 | Sample 7 |
| 0% | 0.5% | 1% | 1.5% | 2% | 2.5% | 3% |

Sample number versus amount of film-coating layer (as percentage relative to core weight)

Light Stability Studies

10 tablets of each sample were placed in a light cabinet (Heraeus Suntest CPS+) and were exposed to light providing an overall illumination of not less than 1.2 million lux hours, and an integrated near ultraviolet energy of not less than 200 watt hours/m². The tablets were placed directly in petri dishes in a single layer. A dark control was used to evaluate the contribution of thermally induced changes to the total change. The dark control was placed beside the sample and was protected against light, wrapped in aluminium foil. After light exposure the tablets (sample and dark control) were analysed for degradation products by HPLC analysis.

Preparation of Samples for HPLC Analysis

The samples of 10 tablets were disintegrated by the means of 0.1 M acetic acid. Next acetonitrile was added for extraction and subsequently the samples were made to volume with methanol/water (50/50). Final concentration: 0.4 mg/ml.

HPLC Analysis

Samples from the light stability studies were analysed by HPLC.

Reversed phase chromatography was performed using a LiChrospher 100 RP-8, 5 μm, 250×4 mm ID with pre-column: LiChrospher 100 RP-8, 5 μm.

The mobile phase consisted of Tetrahydrofurane/acetonitrile/25 mM phosphate buffer pH 7.2 (7/43/50).

25 mM Phosphate Buffer pH 7.2: Dissolve 5.82 g of Na₂HPO₄, 12H₂O and 1.19 g of KH₂PO₄ in 1 litre of water. Adjust the pH by means of NaOH or H₃PO₄.

The flow rate was set at 1.2 ml/min, the column temperature at 40° C., UV detection at UV 230 nm.

Quantification of the degradation products was done from area %, using the normalization factor 0.9 for Lu 26-115 (1-(2-(4-(5-Chloro-1-(4-fluorophenyl)-1H-indol-3-yl)-1-piperidinyl-1-oxide)ethyl)-2-imidazolidinone) and the normalization factor 1.0 for other impurities.

Results

TABLE 4

| | | Degradation as percentage of content of sertindole (Sum %) | | | | | |
|---|---|---|---|---|---|---|---|
| | | Exp. 1 | Exp. 2 | Exp. 3 | Exp. 4 | Exp. 5 | Exp. 6 |
| | Film % of tabl. | Polyethylen | | Polyethylen | | Polyethylen | |
| Sample | Weight | glykol 400 - 0% | | glykol 400 - 4% | | glykol 400 - 8% | |
| Dark control | | 0.094 | NT | 0.085 | 0.1 | 0.095 | 0.087 |
| Sample 1 | 0% | 0.45 | NT | 0.37 | 0.35 | 0.43 | 0.5 |
| Sample 2 | 0.5% | 0.26 | 0.22 | 0.32 | 0.19 | 0.3 | 0.33 |
| Sample 3 | 1% | 0.17 | 0.14 | 0.17 | 0.14 | 0.17 | 0.19 |
| Sample 4 | 1.5% | 0.12 | 0.11 | 0.11 | 0.11 | 0.15 | 0.16 |
| Sample 5 | 2% | 0.11 | 0.11 | 0.12 | 0.1 | 0.1 | 0.12 |
| Sample 6 | 2.5% | 0.1 | 0.11 | 0.12 | 0.091 | 0.12 | 0.12 |
| Sample 7 | 3% | 0.1 | 0.1 | 0.12 | 0.12 | 0.11 | 0.12 |

Sertindole filmcoated tablets 8 mg. Amount of filmcoating layer containing yellow iron oxide (composition refer til table 2) versus amount of degradants (per cent relative to sertindole) after exposure to 1.2 mio Lux.
(NT = not testet)

TABLE 5

| | | Degradation (Sum %) |
|---|---|---|
| | Film % of tabl. | Exp. 7 |
| Sample | Weight | Polyethylen glykol 400 - 4% |
| Dark control | | 0.13 |
| Sample 1 | 0% | 0.37 |
| Sample 2 | 0.5% | 0.35 |
| Sample 3 | 1% | 0.34 |
| Sample 4 | 1.5% | 0.32 |
| Sample 5 | 2% | 0.29 |
| Sample 6 | 2.5% | 0.28 |
| Sample 7 | 3% | 0.28 |

Sertindole filmcoated tablets 8 mg. Amount of filmcoating layer without yellow iron oxide (composition refer til table 2) versus amount of degradants (per cent relative to sertindole) after exposure to 1.2 mio Lux.

TABLE 6
Degradation as percentage of content of sertindole (Sum %)

| | Experiment | | | | | |
|---|---|---|---|---|---|---|
| | Exp. 1 | Exp. 2 | Exp. 3 | Exp. 4 | Exp. 5 | Exp. 6 |
| | Polyethylenglykol 400 | | | | | |
| mcg Iron Oxide | Polyethylen | | Polyethylen | | Polyethylen | |
| Yellow/mm2 | glykol 400 - 0% | | glykol 400 - 4% | | glykol 400 - 8% | |
| Dark control | 0.094 | NT | 0.085 | 0.10 | 0.095 | 0.087 |
| 0.00 | 0.45 | NT | 0.37 | 0.35 | 0.43 | 0.50 |
| 0.12 | 0.26 | 0.22 | 0.32 | 0.19 | 0.30 | 0.33 |
| 0.25 | 0.17 | 0.14 | 0.17 | 0.14 | 0.17 | 0.19 |
| 0.37 | 0.12 | 0.11 | 0.11 | 0.11 | 0.15 | 0.16 |
| 0.50 | 0.11 | 0.11 | 0.12 | 0.10 | 0.10 | 0.12 |
| 0.62 | 0.10 | 0.11 | 0.12 | 0.091 | 0.12 | 0.12 |
| 0.74 | 0.10 | 0.10 | 0.12 | 0.12 | 0.11 | 0.12 |

Sertindole filmcoated tablets 8 mg. Amount of yellow iron oxide per unit tablet surface area (mcg/mm2) (composition refer til table 2) versus amount of degradants (per cent relative to sertindole) after exposure to 1.2 mio Lux.
(NT = not tested)

We claim:

1. A pharmaceutical composition comprising sertindole and a protective agent absorbing radiation with a wavelength below 400 nm.

2. A solid pharmaceutical composition comprising a mixture of sertindole and a protective agent absorbing radiation with a wavelength below 400 nm.

3. The solid pharmaceutical composition of claim 1 or 2 wherein the protective agent absorbing radiation with a wavelength below 400 nm, is present in an amount sufficient to protect the sertindole from light degradation.

4. The solid pharmaceutical composition of any one of claims 1-3 wherein it is selected from tablets and capsules.

5. The solid pharmaceutical composition of any one of claims 1-4 wherein the protective agent absorbing radiation wavelength below 400 nm is selected from iron oxide, such as yellow, red, or black iron oxide as well as mixtures thereof.

6. The solid pharmaceutical composition of any one of claims 1-4 wherein the protective agent absorbing radiation with a wavelength below 400 nm is selected from Carbon black, indigocarmine, indigocarmine- aluminium lake, and permanently or provisionally listed colour additives subject to U.S. certification in 2000 (Based on 21 CFR 2000) absorbing light of wavelength<400 nm, metals or mixture of metals absorbing light of wavelength<400 nm, salts of metals or mixture of metal salts absorbing light of wavelength<400 nm

7. The solid pharmaceutical composition of any one of claims 1-6 wherein the protective agent absorbing radiation with a wavelength below 400 nm is present in an amount up to 5% relative to the weight of the solid composition.

8. The solid pharmaceutical composition of any one of claims 1-7 further comprising a coating, such as a film-coating layer.

9. The solid pharmaceutical composition of claim 8 wherein the coating layer comprises titanium dioxide, such as titanium dioxide in an amount of at least 0.5 weight % of dry substance in the coating layer.

10. A solid pharmaceutical composition comprising sertindole and a coating layer comprising a protective agent absorbing radiation with a wavelength below 400 nm.

11. The solid pharmaceutical composition of claim 10 wherein the protective agent absorbing radiation with a wavelength below 400 nm, is present in an amount sufficient to protect the sertindole from light degradation.

12. The solid pharmaceutical composition of any one of claims 10-11 wherein it is selected from tablets and capsules.

13. The solid pharmaceutical composition of any one of claims 10-12 wherein the protective agent absorbing radiation wavelength below 400 nm is selected from iron oxide, such as yellow, red, or black iron oxide as well as mixtures thereof.

14. The solid pharmaceutical composition of any one of claims 10-12 wherein the protective agent absorbing radiation with a wavelength below 400 nm is selected from Carbon black, indigocarmine, indigocarmine-aluminium lake, and permanently or provisionally listed colour additives subject to U.S. certification in 2000 (Based on 21 CFR 2000) absorbing light of wavelength<400 nm, metals or mixture of metals absorbing light of wavelength<400 nm, salts of metals or mixture of metal salts absorbing light of wavelength<400 nm.

15. The solid pharmaceutical composition of any one of claims 10-14 wherein the coating layer further comprises titanium dioxide, such as titanium dioxide in an amount of at least 0.5 weight % of dry substance in the coating layer.

16. The solid pharmaceutical composition of any one of claims 10-15 wherein the coating layer comprises iron oxide, such as iron oxide in an amount of at least 0.1 μg/mm² surface area of the composition.

17. The solid pharmaceutical composition of any one of claims 10-16 wherein the coating layer is less than 20% weight relative to the core of the composition.

18. The solid pharmaceutical composition of any one of claims 10-17 wherein the coating layer further comprises a plasticizer, such as one or more of polyethylene glycol, triethyl citrate, triacetin, acetyl triacetylcitrate, acetyltributyl citrate, dibutyl phthalate, dibutyl sebacate, diethyl phthalate, tributyl citrate.

19. The solid pharmaceutical composition of any one of claims 10-18 wherein the coating layer further comprises a polyethylene glycol in an amount of less than 12 weight % relative to the coating layer, such as less than 8 weight %.

20. The solid pharmaceutical composition of any one of claims 1-19 wherein the amount of sertindole in the composition is from 1 mg to 48 mg, such as 2 mg to 24 mg.

21. Use of sertindole for the preparation of a pharmaceutical composition comprising sertindole and a coating layer comprising a protective agent absorbing radiation with a wavelength below 400 nm, such as iron oxide, for the treatment of schizophrenia.
